(19) United States
(12) Patent Application Publication
(10) Pub. No.: US 2020/0022629 A1
(43) Pub. Date: Jan. 23, 2020

(54) SKIN PRICK TEST DEVICE

(71) Applicant: Hance MA, Henan (CN)

(72) Inventor: Hance MA, Henan (CN)

(21) Appl. No.: 16/059,069

(22) Filed: Aug. 9, 2018

(30) Foreign Application Priority Data
Jul. 18, 2018 (CN) 201810787556.8

(51) Int. Cl. A61B 5/15; B67D 3/00; A61B 5/00

(52) U.S. Cl. CPC A61B 5/150167; A61B 5/150412; A61B 5/15146; A61B 5/411; A61B 5/150358; B67D 3/0032

(57) ABSTRACT

The present invention relates to the field of test devices. The skin prick test device according the present invention mainly comprises conjoined bottles, a tray and pricking devices, wherein each set of conjoined bottles, which comprises a large bottle body and a small bottle body, is arranged in clamping holes of the tray, and liquid medicine can flow into the small bottle body from the large bottle body through an inclined surface, thereby avoiding frequent refilling of the liquid medicine. In addition, each connecting base has a trapezoidal cross section with its bottom surface being flush with that of the holder, and is gradually widened from the top down to fit with the parallel clamp plates on the tray, so that the pricking device has better stability as both ends thereof are prevented from excessively shaking longitudinally when the pricking device is taken out of and put into the tray.

### BACKGROUND OF THE INVENTION

The present invention relates to the field of test devices, in particular to a skin prick test device.

Skin prick test is recognized as the most convenient, economical, safe, effective and also the most commonly used allergen diagnosis method in the medical field. Such skin prick test is carried out on the skin at palmar side of the forearm of a patient, including the following specific operation procedures of: firstly, after the routine skin disinfection, dropping a drop of allergen test solution, a drop of negative control solution and a drop of positive control solution onto the cleaned skin from the top down, with a distance between the two liquid drops being 3-5 cm; secondly, vertically penetrating a puncture needle through the liquid drops and into the skin when the patient's skin is tight; and finally, observing the test results 10-20 minutes later after pricking, and determining the patient's response intensity to the allergen by comparing urticaria sizes of the allergen and the positive control.

In the process of using existing products, the puncture needle of the pricking device is easy to pick up too much liquid medicine when dipped, resulting in great waste. It is found in experiments that the amount of liquid medicine covered on the puncture needle is related to the diameter of a liquid medicine container, i.e., the closer the wall of a liquid medicine bottle is to the pricking device, the less likely the puncture needle on the pricking device is to pick up the liquid medicine due to the effect of the surface tension of liquid. However, if the diameter of the medicine bottle is too small, frequent refilling of the liquid medicine is required, thus causing trouble.

### BRIEF SUMMARY OF THE INVENTION

In order to solve the above problems, the present invention provides a skin prick test device, which makes the puncture needles on the pricking device not easy to pick up too much liquid medicine when dipped without causing any waste, and also does not need frequent refilling of the liquid medicine into medicine bottles at the same time.

In order to achieve the above objective, a technical solution adopted by the present invention is as follows: a skin prick test device is provided, including conjoined bottles, a tray and pricking devices, wherein clamping holes having the same shape as the conjoined bottles are arranged at an upper end of the tray, each set of conjoined bottles, which includes bottle caps and bottle bodies, is arranged in the clamping holes; the bottle bodies comprise a first bottle body and a second bottle body which are of an integrated structure and internally communicated, and the first bottle body has a radius smaller than that of the second bottle body; each bottle body is provided with a pricking cavity inside and an opening at its upper end, the bottle cap is clamped and fixed to the opening at the upper end of the bottle body and is provided with a through hole corresponding to the position of the first bottle body, and the through hole is communicated with the pricking cavity; and protruding parallel clamp plates to which the pricking device is clamped are also arranged on an upper end surface of the tray.

Further, the pricking device includes a holder having symmetrical connecting bases uniformly arranged on both sides thereof respectively, and prickers which are fixed at a lower end of each connecting base; puncture needles are arranged at a lower end of each pricker, and the holder is clamped to the parallel clamp plates.

A circular clamping cap is also arranged between the connecting base and the pricker.

Rectangular grooves convenient to hold by hands are also uniformly arranged in a staggered manner on both sides of the holder.

The parallel clamp plates include first parallel clamp plates whose height is greater than that of the holder, and second parallel clamp plates whose height is greater than that of the pricker.

Further, each set of parallel clamp plates comprises two protruding clamp plates which are parallel to each other, and a clamping groove, in which the pricking device is clamped, is formed between the protruding clamp plates.

Further, an inner bottom surface of the second bottle body is higher than that of the first bottle body, and an inclined surface is arranged between the inner bottom surfaces of the second bottle body and the first bottle body.

Further, the bottle bodies are clamped and fixed to the clamping holes, or form an integrated structure with the clamping holes.

Further, each connecting base has a trapezoidal cross section with its bottom surface being flush with that of the holder.

The conjoined bottles are symmetrically arranged with the parallel clamp plates as the center.

The beneficial effects of the present invention are as follows: The skin prick test device according the present invention mainly includes the conjoined bottles, the tray and the pricking devices, wherein each set of the conjoined bottles, which includes the large bottle body and the small bottle body, is arranged in clamping holes of the tray, and liquid medicine can flow into the small bottle body from the large bottle body through the inclined surface, thereby avoiding frequent refilling of the liquid medicine. Meanwhile, the pricking device dips in the liquid medicine in the small bottle body, so that the waste of liquid medicine can be minimized to achieve the purpose of saving the liquid medicine. Parallel clamp plates at the upper end of the tray can accurately position the holder of the pricking device on the tray and prevent the holder from shaking to fix and guide the holder at the same time. In addition, each connecting base has a trapezoidal cross section with its bottom surface being flush with that of the holder, and is gradually widened from the top down to fit with the parallel clamp plates on the tray, so that the pricking device has better stability as both ends thereof are prevented from excessively shaking longitudinally when the pricking device is taken out of and put into the tray.

### BRIEF DESCRIPTION OF THE DRAWINGS

FIG. 1 is a structural diagram of the present invention;

FIG. 2 is an exploded view of a conjoined bottle of the present invention;

FIG. 3 is a three-dimensional diagram of the conjoined bottle of the present invention;

FIG. 4 is a three-dimensional diagram of a tray of the present invention;

FIG. 5 is a three-dimensional diagram of a pricking device of the present invention; and

FIG. 6 is a top view of the pricking device of the present invention, in which:

1: conjoined bottle; 11: bottle cap; 111: through hole; 12: bottle body; 121: first bottle body; 122: second bottle body; 123: opening; 124: inclined surface; 2: tray; 21: clamping hole; 22: parallel clamp plate; 221: first parallel clamp plate; 222: second parallel clamp plate; 223: clamping groove; 3: pricking device; 31: holder; 311. rectangular groove; 32: pricker; 33: connecting base; 34: puncture needle; 35: circular clamping cap.

### DETAILED DESCRIPTION OF THE INVENTION

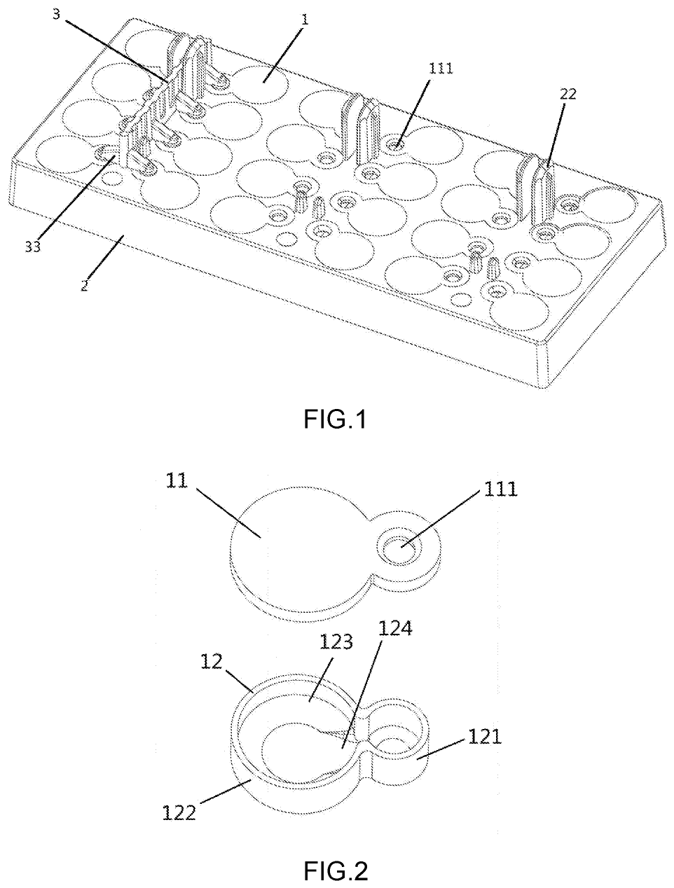

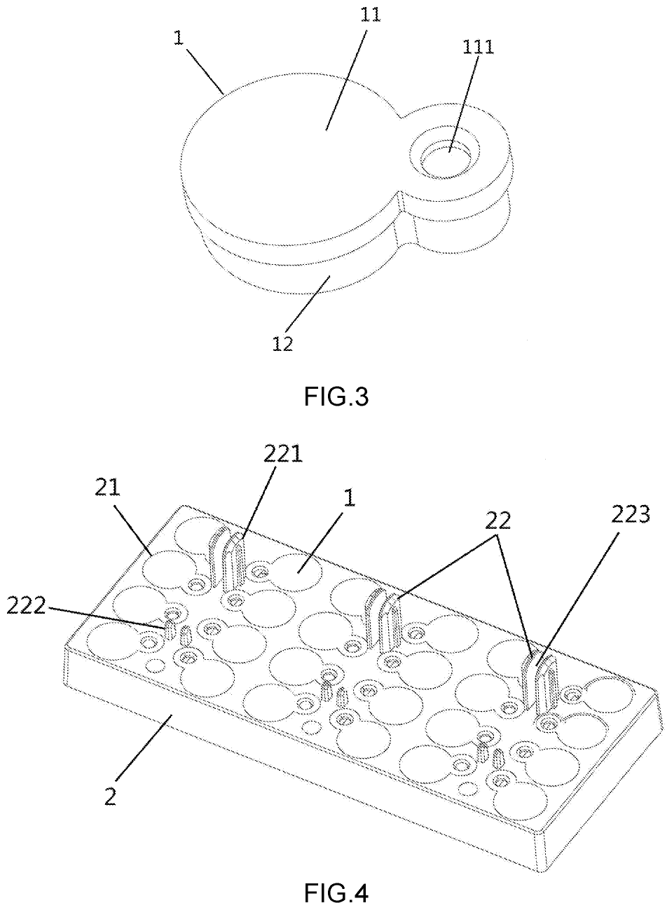

Referring to FIGS. 1 to 4, the present invention relates to a skin prick test device, including conjoined bottles 1, a tray 2 and pricking devices 3, wherein clamping holes 21 having the same as the conjoined bottles 1 are arranged at in upper end of the tray 2, each set of conjoined bottles 1, which includes bottle caps 11 and bottle bodies 12, is arranged in the clamping holes 21; the bottle bodies 12 comprise a first bottle body 121 and a second bottle body 122 which are of an integrated structure and internally communicated, and the first bottle body 121 has a radius smaller than that of the second bottle body 122; each bottle body 12 is provided with a pricking cavity inside and an opening 123 at its upper end, the bottle cap 11 is clamped and fixed to the opening 123 at the upper end of the bottle body 12 and is provided with a through hole 111 corresponding to the position of the first bottle body 121, and the through hole 111 is communicated with the pricking cavity; and protruding parallel clamp plates 22 to which the pricking device 3 is clamped are also arranged on an upper end surface of the tray 2.

Compared with the prior art, the skin prick test device according the present invention mainly includes conjoined bottles 1, a tray 2 and pricking devices 3, wherein each set of conjoined bottles 1, which includes a first small bottle body 121 and a second large bottle body 122, is clamped and fixed in clamping holes 21 of the tray 2. Liquid medicine can flow into the small bottle body from the large bottle body through an inclined surface, thereby avoiding frequent refilling of the liquid medicine frequently. Meanwhile, the pricking device 3 dips in the liquid medicine in the small bottle body, so that the waste of liquid medicine can be minimized to achieve the purpose of saving the liquid medicine.

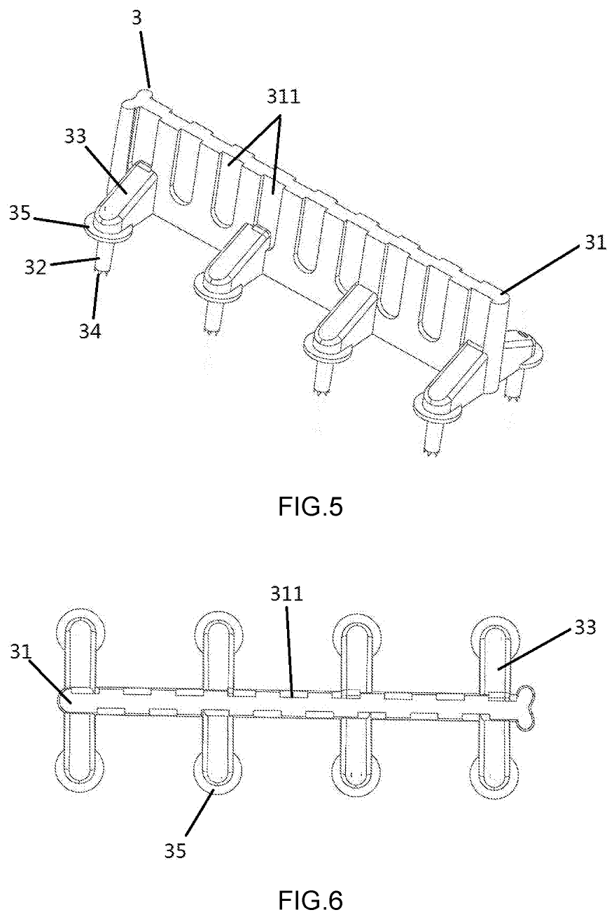

Further, referring to FIGS. 5 to 6, the pricking device 3 includes a holder 31 having symmetrical connecting bases 33 uniformly arranged on both sides thereof respectively, and prickers 32 which are fixed at a lower end of each connecting base 33; puncture needles 34 are arranged at a lower end of each pricker 32, and the holder 31 is clamped to parallel clamp plates 22.

With the above solution, the parallel clamp plates 22 at an upper end of the tray 2 can accurately position the holder 31 of the pricking device 3 on the tray 2 and prevent the holder from shaking to fix and guide the holder at the same time.

A circular clamping cap 35 is also arranged between the connecting base 33 and the pricker 32. The circular clamping cap 35 is matched with the through hole 111 in the bottle cap 11 in shape, and the bottle cap is closed by matching the circular clamping cap 35 with the through hole 111 in the bottle cap 11 to prevent waste caused by evaporation of the liquid medicine, thereby saving the liquid medicine.

Rectangular grooves 311 convenient to hold by hands are uniformly arranged in a staggered manner on both sides of the holder 31. With the above structure, a structure that protrusions and recesses are arranged in a staggered manner on the opposite sides of the holder 31 is formed. Such structure is most favorable for gripping as the protrusions and the recesses are opposite and have the same width, so that the holder 31 has a better hand feeling and is beneficial for the user to grip the pricking device 3.

The parallel clamp plates 22 include first parallel clamp plates 221 whose height is greater than that of the holder 31, and second parallel clamp plates 222 whose height is greater than that of the pricker 32.

Further, each set of parallel clamp plates 22 includes two protruding clamp plates which are parallel to each other, and a clamping groove 223 in which the pricking device 3 is clamped is formed between the protruding clamp plates.

With the above solution, the holder 31 of the pricking device 3 is accurately positioned on the tray 2 by two sets of parallel clamping plates 22 so that the puncture needles 34 of the pricking device 3 can accurately pass through the through hole 111 at an upper end of the first bottle body 121. The first parallel clamp plates 221 on a front side of the tray 2, together with the connecting base 33, can prevent front and rear parts of the pricking device 3 from shaking longitudinally, and together with the second parallel clamp plates 222, can prevent the pricking device 3 from shaking laterally. The second clamp plates 222 has a height greater than the length of the pricker 32 of the pricking device 3, to prevent the puncture needle 34 of the pricking device 3 from touching the surface of the tray 2 when the pricking device 3 is taken out and put in.

Further, an inner bottom surface of the second bottle body 122 is higher than that of the first bottle body 121, and an inclined surface 124 is arranged between the inner bottom surfaces of the second bottle body 122 and the first bottle body 121.

With the above solution, the liquid medicine can flow into the first bottle body 121 from the second bottle body 122 through the inclined surface 124, and is finally picked up in the first bottle body 121 by the pricking device 3.

In this embodiment, the puncture needle 34 of the pricking device 3 enters the first bottle body 121 through the through hole 111 to dip in the liquid medicine. As the diameter of the first bottle body 121 is very small, the bottle wall is very close to the pricker 32 of the pricking device 3, and when the pricking device 3 is taken out, the liquid medicine on the pricker 32 of the pricking device 3 will be pulled off by the bottle wall due to the effect of the surface tension of liquid, so that the waste of liquid medicine can be minimized to achieve the purpose of saving the liquid medicine.

Further, the bottle bodies 12 are clamped and fixed to the clamping holes 21, or form an integrated structure with the clamping holes 21.

In this embodiment, the bottle bodies 12 of the conjoined bottles 1 may be separated from the clamping holes 21 of the tray 2 and taken out separately, or may be processed into an integrated structure so that the bottle bodies 12 cannot be taken out of the clamping holes 21.

Further, each connecting base 33 has a trapezoidal cross section with its bottom surface being flush with that of the holder.

With the above solution, each connecting base 33 is gradually widened from the top down to fit with the parallel clamp plates 22 on the tray 2, so that the pricking device 3 has better stability as both ends thereof are prevented from excessively shaking longitudinally when the pricking device 3 is taken out of and put into the tray 2.

The conjoined bottles 1 are symmetrically arranged with the parallel clamp plates 22 as the center.

In this embodiment, the first bottle body 121 of the conjoined bottles 1 is a small bottle body and the second bottle body 122 is a large bottle body, wherein the second bottle body 122 is sufficiently large in volume to ensure that multiple times of pricking can be provided, and the liquid medicine in the second bottle body 122 will flow into the first bottle body 121, while the first bottle body 121 is sufficiently small in diameter to ensure that the pricking device 3 does not pick up too much liquid medicine when dipped, and its bottle wall can filter out the excess liquid medicine on the pricker 32 of the pricking device 3 due to the effect of the surface tension of liquid when the pricking device 3 is taken out of the tray 2, thereby avoiding waste of the liquid medicine.

In this embodiment, the holder of the pricking device 3 is a cuboid holder 31 having four connecting bases 33 respectively arranged on its both sides, and a lower end of each connecting base 33 is connected to the pricker 32, and a lower end of the pricker 32 is connected to a plurality of puncture needles 34 which can dip in the liquid medicine under the action of surface tension of liquid. Meanwhile, the tray 2 is provided with three sets of clamping hole structures which can hold three groups of pricking devices 3 at the same time, and each set of clamping hole structures is provided with four sets of symmetrical clamping holes 21 matched with the prickers 32 on the holder 31.

The above embodiments merely describe the preferred embodiments of the present invention, and are not intended to limit the scope of the present invention. Without departing from the spirit of the present invention, various modifications and improvements made by a person of ordinary skill in the art to the technical solution of the present invention shall all fall within the protection scope defined by the claims of the present invention.

What is claimed is:

1. A skin prick test device, comprising conjoined bottles, a tray and pricking devices, wherein clamping holes having the same as the conjoined bottles are arranged at an upper end of the tray, each set of conjoined bottles, which comprises bottle caps and bottle bodies, is arranged in the clamping holes; the bottle bodies comprise a first bottle body and a second bottle body which are of an integrated structure and internally communicated, and the first bottle body has a radius smaller than that of the second bottle body; each bottle body is provided with a pricking cavity inside and an opening at its upper end, the bottle cap is clamped and fixed to the opening at the upper end of the bottle body and is provided with a through hole corresponding to the position of the first bottle body, and the through hole is communicated with the pricking cavity; and protruding parallel clamp plates to which the pricking device is clamped are also arranged on an upper end surface of the tray.

2. The skin prick test device according to claim 1, wherein the pricking device comprises a holder having symmetrical connecting bases uniformly arranged on both sides thereof respectively, and prickers which are fixed at a lower end of each connecting base; puncture needles are arranged at a lower end of each pricker, and the holder is clamped to the parallel clamp plates.

3. The skin prick test device according to claim 2, wherein a circular clamping cap is also arranged between the connecting base and the pricker.

4. The skin prick test device according to claim 2, wherein rectangular grooves convenient to hold by hands are uniformly arranged in a staggered manner on both sides of the holder.

5. The skin prick test device according to claim 2, wherein the parallel clamp plates comprise first parallel clamp plates whose height is greater than that of the holder, and second parallel clamp plates whose height is greater than that of the pricker.

6. The skin prick test device according to claim 1, wherein each set of parallel clamp plates comprises two protruding clamp plates which are parallel to each other, and a clamping groove, in which the pricking device is clamped, is formed between the protruding clamp plates.

7. The skin prick test device according to claim 1, wherein an inner bottom surface of the second bottle body is higher than that of the first bottle body, and an inclined surface is arranged between the inner bottom surfaces of the second bottle body and the first bottle body.

8. The skin prick test device according to claim 1, wherein the bottle bodies are clamped and fixed to the clamping holes, or form an integrated structure with the clamping holes.

9. The skin prick test device according to claim 1, wherein each connecting base has a trapezoidal cross section with its bottom surface being flush with that of the holder.

10. The skin prick test device according to claim 1, wherein the conjoined bottles are symmetrically arranged with the parallel clamp plates as the center.
